(19) United States
(12) Patent Application Publication
(10) Pub. No.: US 2024/0050761 A1
(43) Pub. Date: Feb. 15, 2024

(54) SYSTEM AND METHODS FOR INDUCTIVE PULSE BURST TREATMENT

(71) Applicant: HUMANITY NEUROTECH, INC., Oakland, CA (US)

(72) Inventor: Blake GURFEIN, Oakland, CA (US)

(21) Appl. No.: 18/258,023

(22) PCT Filed: Dec. 20, 2021
(86) PCT No.: PCT/US2021/064453

§ 371 Date: Jun. 16, 2023

Related U.S. Application Data
(60) Provisional application No. 63/127,153, filed on Dec. 18, 2020.

(51) Int. Cl. A61N 2/00; A61N 2/02

(52) U.S. Cl. CPC A61N 2/006; A61N 2/02

(57) ABSTRACT

Apparatuses and methods for delivery of pulsed electromagnetic field (PEMF) energy therapy to modulate inflammation using high-frequency current pulsed through one or more coils. For example, described herein are pulse burst waveforms, coil applicators for the head and body, and implantable biocompatible coil arrays. The methods and apparatuses described herein include parameters for PEMF energy that specifically and robustly reduce an inflammatory response. Also described herein are methods and apparatuses increasing an inflammatory response using PEMF parameters that specifically and robustly increase in inflammatory response.

### CLAIM OF PRIORITY

This patent application claims priority to U.S. Provisional Patent Application No. 63/127,153, filed Dec. 18, 2020, titled “SYSTEM AND METHODS FOR INDUCTIVE PULSE BURST TREATMENT” and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Inductive neuromodulation typically involves generating an electromagnetic field by passing current through an antenna (e.g., coil), and applying the resulting electromagnetic field to non-invasively modulate the activity of nervous system tissue adjacent to the antenna (e.g., coil). In contrast to transcranial electrical stimulation (TES), inductive neuromodulation approaches such as transcranial magnetic stimulation (hereinafter “TMS”) can emit electromagnetic fields that range from weak to strong enough to induce neuronal action potentials in isolation and have the advantage of well-defined physics that dictate exactly what regions of brain or nerve tissue are being affected. In addition to TMS, other inductive neuromodulation systems have been developed and are generically referred to as pulsed electromagnetic field (hereinafter “PEMF”) emitting devices or low field magnetic stimulation (hereinafter “LFMS”) devices. Inductive neuromodulation systems have been disclosed, including TMS (see for example Miller U.S. Pat. No. 6,926,660; Zangen et al. U.S. Pat. No. 8,277,371; Simon et al. U.S. Pat. No. 8,972,004; Schneider et al. U.S. Pat. No. 8,267,850), PEMF (see for example Pilla et al. U.S. Pat. Nos. 9,440,089, 9,433,797, 9,427,598 and US Patent application 2014/354,587; Cadossi et al. US Patent application 2014/657,365), and LFMS systems (see for example Phillips et al. U.S. Pat. No. 9,713,729; Rohan et al. U.S. Pat. No. 7,282,021).

Inductive neuromodulation technologies can be broken down into those that produce supra-threshold fields strong enough to induce action potentials and those that produce weaker sub-threshold fields. TMS devices typically emit supra-threshold electromagnetic fields and exert their neuromodulatory effects through repeatedly passing current through coils positioned adjacent to the skull, thereby activating neuronal firing at a specific frequency in a specific locus. Sub-threshold inductive neuromodulation devices such as PEMF and LFMS are similar to TMS in terms of dependence on coil-based induction, however these approaches often produce weaker sub-threshold fields and are typically applied to a large area of tissue rather than a well-defined target region. These devices range in design and waveform specifications, but frequently employ large coils that are capable of treating large regions of the brain, for example devices intended to treat stroke and depression. See, e.g., Capone 2017, Rohan 2014.

Sub-threshold inductive approaches like PEMF and LFMS do not emit fields strong enough to evoke action potentials, however they may increase the likelihood of neuronal firing via partial depolarization. Importantly, the application of electromagnetic fields spanning sub-threshold to supra-threshold field strengths may also have important biological effects unrelated to depolarization.

Although PEMF has been suggested to act by reducing inflammation, to date it has proven difficult to reproducibly achieve a robust reduction in inflammation. It would be beneficial to provide methods and apparatuses for modulating an inflammatory response using PEMF. The methods and apparatuses described herein may address these needs.

### SUMMARY OF THE DISCLOSURE

Described herein are methods and apparatuses (e.g., systems, devices, etc.) for the application of pulsed electromagnetic (PEMF) energy for treatment of a patient or a tissue of a patient. In particular, described herein are PEMF methods and apparatuses (e.g., devices, systems, etc.) for the application of PEMF having parameters that can reduce an inflammatory response (e.g., reduce inflammation) in a patient or a specific tissue of a patient. Surprisingly, the inventor has found that the inflammatory response of cells, tissues and patients (including animals) to PEMF is strongly dependent on the applied duty cycle and amplitude and has, for the first time, identified a critical range within which a strong effect on inflammation is seen, as identified by examining levels of cytokines/chemokines. Outside of the identified range, only a very small, and/or transient effect, or no effect at all, is seen; however within a second range an opposite effect is consistently and significantly observed, in which the inflammatory response is increased. Significantly commercially available PEMF apparatuses typically operate outside of these ranges.

These methods and apparatuses may, in some examples, provide non-invasive, inductive delivery of PEMF energy to the central and peripheral nervous system and other bodily tissues. The methods and apparatuses described herein may be delivered systemically to the patient or in a targeted manner, delivering PEMF energy to a particular tissue or region of tissue (e.g., head, skin, organ, etc.). In particular, the apparatuses and methods for inductive delivery of energy (PEMF energy) to tissue described herein may induce effects by passing specific waveforms through one or more applicators (e.g., coils, antenna, etc., generically described as “coils” herein) to promote therapeutic, wellness, or performance-enhancing effects. For example, these systems and methods for inductive treatment may include: (1) pulse burst waveforms; (2) coil applicators for the head and body; (3) implantable biocompatible coil arrays; (4) integration of electroencephalography for closed-loop stimulation; (5) artificial intelligence algorithms for optimizing coil position and stimulation paradigm; (6) a closed-loop user interface that is operated via smartphone, tablet, or virtual reality headset.

Described herein are method and apparatuses for decreasing or eliminating an inflammatory response. In general, a method of reducing inflammation in a patient may include: applying pulsed electromagnetic energy to the patient from a coil, wherein the pulsed electromagnetic energy has a duty cycle of 4% or greater (e.g., between 4% and 30%, between 4% and 25%, between 4% and 22%, between 4% and 20%, between 4% and 19%, between 4% and 18%, between 4% and 17%, between 4% and 16%, between 4% and 15%, between 4% and 14%, between 4% and 13%, between 4% and 12%, between 4% and 11%, between 4% and 10%, between 4% and 9%, between 4% and 8%, between 4% and 7%, between 3% and 20%, between 3% and 35%, between 3% and 15%, etc.) and an amplitude of between 0.2 G and 1 G (or between 0.2 G and less than 1 G, between 0.2 G and 0.95 G, between 0.2 G and 0.9 G, between 0.2 G and 0.85 G, between 0.21 G and 1 G, between 0.22 G and 1 G, between 0.23 G and 1 G, between 0.24 G and 1 G, between 0.21 G and less than 1 G, between 0.21 G and 0.95 G, between 0.21 G and 0.9 G, etc.), resulting in a decrease in an inflammatory response by the patient (or by a target patient tissue that has been treated). For example, the decrease in inflammatory response may be a decrease in one or more cytokine and/or chemokine. In particular, the decrease in inflammatory response may be a decrease of about 20% or more (e.g., 25% or more, 27% or more, 30% or more, 35% or more, 40% or more, 45% or more, 50% or more, etc.) of a cytokine or chemokine. In some examples the decrease may be in one or more of: Tumor Necrosis Factor alpha (TNF-α), Macrophage Inflammatory Protein (MIP1a), Interleukin 1 beta (IL-1β), Interferon gamma-induced protein 10 (IP-10), and Monocyte Chemoattractant Protein-1 (MCP-1).

For example, a method of increasing inflammation in a patient may include: applying pulsed electromagnetic energy to the patient from a coil for more than 10 minutes, wherein the pulsed electromagnetic energy has a duty cycle of 4% or greater and an amplitude of greater than 1 G, resulting in an increase in inflammation in the patient.

A method of reducing inflammation in a target tissue may include: applying pulsed electromagnetic energy from a coil adjacent to the target tissue for more than 10 minutes, the pulsed electromagnetic energy having a duty cycle of 4% or greater and an amplitude of between 0.2 G and 0.9 G, resulting in a decrease in the target tissue of 30% or more of one or more of: Tumor Necrosis Factor alpha (TNF-α), Macrophage Inflammatory Protein (MIP1a), Interleukin 1 beta (IL-1β), Interferon gamma-induced protein 10 (IP-10), and Monocyte Chemoattractant Protein-1 (MCP-1).

A method of reducing inflammation in a patient may include: applying pulsed electromagnetic energy to the patient from a coil, wherein the pulsed electromagnetic energy has a duty cycle of 4% or greater and an amplitude of between 0.2 G and 1 G, resulting in a decrease in the patient of 30% or more of one or more of: Tumor Necrosis Factor alpha (TNF-α), Macrophage Inflammatory Protein (MIP1a), Interleukin 1 beta (IL-1β), Interferon gamma-induced protein 10 (IP-10), and Monocyte Chemoattractant Protein-1 (MCP-1).

In some examples, a method of reducing inflammation in a target tissue may include: applying pulsed electromagnetic energy from a coil adjacent to the target tissue, the pulsed electromagnetic energy having a duty cycle of 4% or greater and an amplitude of between 0.2 G and 1 G, resulting in a decrease in the target tissue of 30% or more of one or more of: Tumor Necrosis Factor alpha (TNF-α), Macrophage Inflammatory Protein (MIP1a), Interleukin 1 beta (IL-1β), Interferon gamma-induced protein 10 (IP-10), and Monocyte Chemoattractant Protein-1 (MCP-1).

In any of these methods, applying pulsed electromagnetic energy may include applying for at least 10 minutes. In some examples, the applied pulsed electromagnetic energy may have a duty cycle of between about 4% and about 20%, as mentioned above.

Applying the pulsed electromagnetic energy may include decreasing levels of one or more of: Tumor Necrosis Factor alpha (TNF-α), Macrophage Inflammatory Protein (MIP1a), Interleukin 1 beta (IL-1β), Interferon gamma-induced protein 10 (IP-10), and Monocyte Chemoattractant Protein-1 (MCP-1), by 40% or more in the target tissue.

Any of these methods may include detecting measuring a level of a cytokine or chemokine, such as one or more of: Tumor Necrosis Factor alpha (TNF-α), Macrophage Inflammatory Protein (MIP1a), Interleukin 1 beta (IL-1β), Interferon gamma-induced protein 10 (IP-10), and Monocyte Chemoattractant Protein-1 (MCP-1) from the target tissue.

Applying pulsed electromagnetic energy may include applying busts of pulsed electromagnetic energy having a burst width of between about 10 msec and 500 msec (e.g., 10 msec and 450 msec, 10 msec and 400 msec, 10 msec and 350 msec, 10 msec and 300 msec, 10 msec and 200 msec, 10 msec and 150 msec, 10 msec and 125 msec, 10 msec and 100 msec, 25 msec and 500 msec, 25 msec and 400 msec, 25 msec and 300 msec, 25 msec and 200 msec, etc.).

In any of these methods and apparatuses, the pulsed electromagnetic energy may have a carrier frequency of about 27.12 MHz.

In general, these methods and apparatuses for using them may be non-thermal; thus, the pulsed electromagnetic energy may be applied to the target tissue without substantially increasing a temperature of the target tissue (e.g., increasing it less than 0.5 degrees C., less than 0.4 degrees C., less than 0.3 degrees C., less than 0.2 degrees C., less than 0.1 degrees C., etc.).

In any of these methods applying pulsed electromagnetic energy from the coil adjacent to the target tissue may comprise applying from a plurality of coils adjacent a patient's head. For example, applying pulsed electromagnetic energy from the coil may comprise applying pulsed electromagnetic energy from a plurality of coils coupled to a headgear configured to be worn by a patient.

Applying pulsed electromagnetic energy from the coil may comprise applying pulsed electromagnetic energy from a plurality of coils that are fired independently, simultaneously, and/or in sequence. Applying pulsed electromagnetic energy from the coil(s) may comprises modifying a location or energy applied by the coil(s) based on electroencephalography data acquired before or after application of pulsed electromagnetic energy.

The methods described herein for decreasing an inflammatory response may generally be used to treat, prevent or ameliorate any inflammatory disorder. For example, described herein are methods for reducing or preventing an inflammatory response that may be methods of treating a neuroinflammatory disorder including one or more of: Encephalitis, Myelitis, Meningitis, Arachnoiditis, Neuritis, Dacryoadenitis, Scleritis, Episcleritis, Keratitis, Retinitis, Chorioretinitis, Blepharitis, Conjunctivitis, Uveitis, Otitis externa, Otitis media, Labyrinthitis, and Mastoiditis. In some examples the methods described herein are methods of treating a cardiovascular inflammatory disorder including one or more of: Carditis, Endocarditis, Myocarditis, Pericarditis, Vasculitis, Arteritis, Phlebitis, and Capillaritis. In some examples the methods described herein are methods of treating a respiratory inflammatory disorder including one or more of: Sinusitis, Rhinitis, Pharyngitis, Laryngitis, Tracheitis, Bronchitis, Bronchiolitis, Pneumonitis, Pleuritis, and Mediastinitis. In some examples the methods described herein are methods of treating a digestive system inflammatory disorder including one or more of: Stomatitis, Gingivitis, Gingivostomatitis, Glossitis, Tonsillitis, Sialadenitis/Parotitis, Cheilitis, Pulpitis, Gnathitis, Esophagitis, Gastritis, Gastroenteritis, Enteritis, Colitis, Enterocolitis, Duodenitis, Ileitis, Caecitis, Appendicitis, and Proctitis. For example, the methods described herein may be methods of treating irritable bowel syndrome (IBD).

The methods described herein may be methods of treating one or more of: Hepatitis, Ascending cholangitis, Cholecystitis, Pancreatitis, Peritonitisg, Dermatitis, Folliculitis, Cellulitis, and Hidradenitis, Arthritis, Dermatomyositis, Myositis, Synovitis/Tenosynovitis, Bursitis, Enthesitis, Fasciitis, Capsulitis, Epicondylitis, Tendinitis, Panniculitis, Osteochondritis, (Osteitis/Osteomyelitis), Spondylitis, Periostitis, Chondritis, Nephritis, Glomerulonephritis, Pyelonephritis, Ureteritis, Cystitis, Urethritis, Oophoritis, Salpingitis, Endometritis, Parametritis, Cervicitis, Vaginitis, Vulvitis, Mastitis, Orchitis, Epididymitis, Prostatitis, Seminal vesiculitis, Balanitis, Posthitis, Balanoposthitis, Chorioamnionitis, Funisitis, Omphalitis, Insulitis. Hypophysitis, Thyroiditis, Parathyroiditis, Adrenalitis, Lymphangitis, and Lymphadenitis.

Also described herein are methods of treating inflammation associated with cancer and/or chemotherapy and/or an immunotherapy, including a cancer immunotherapy.

Also described herein are methods of treating a patient having a Severe Acute Respiratory Syndrome coronavirus 2 (SARS-CoV-2) infection.

In some of the examples described herein are methods of modulating inflammation using PEMF by an implantable applicator. For example, any of these methods may include applying PEMF from one or more coils that are implanted into a patient's body.

In general, any of these methods may adjust the applied PEMF based on feedback, including feedback on the effectiveness of prior PEMF applied and/or feedback based on a current or past level of one or more cytokines and/or chemokines. For example, any of these methods may include adjusting the pulsed electromagnetic energy based on a level of one or more of: Tumor Necrosis Factor alpha (TNF-α), Macrophage Inflammatory Protein (MIP1a), Interleukin 1 beta (IL-1β), Interferon gamma-induced protein 10 (IP-10), and Monocyte Chemoattractant Protein-1 (MCP-1).

Also described herein are apparatuses configured to perform any of these methods. For example, an electromagnetic treatment apparatus for reducing inflammation in a target tissue, the apparatus comprising: one or more applicator loops; a frame configured to secure the one or more applicator loops adjacent to a target tissue; and a waveform generator coupled to the one or more applicator loops, the waveform generator comprising a pulse generator configured to deliver a pulsed electromagnetic energy from the one or more applicator loops, the pulsed electromagnetic energy having a duty cycle limited to between 4% and 20% and an amplitude limited to be between 0.2 G and 0.9 G.

The one or more applicator loops may comprise an array of applicator loops (also referred to herein as “coils”). The waveform generator may be configured to deliver the pulsed electromagnetic energy for more than 10 minutes. The frame may comprise a helmet. In some examples, the frame comprises a brace.

Any of the apparatuses described herein may also include a thermal sub-system (e.g., a thermal control subsystem) that includes a temperature sensor configured to monitor a temperature of the target tissue and/or the applicator loop(s), and to apply cooling if the temperature exceeds a threshold. The thermal sub-system may be part of the controller and may include a control loop to modify the dose parameters to prevent or reduce further heating in the target tissue (e.g., if the temperature exceeds the threshold or a separate threshold).

The thermal sub-system may include one or more cooling mechanism, including, for example, a Peltier device for cooling the patient (e.g., via a thermal transfer pad), and/or for cooling the one or more applicators, which may also head up during use.

Although PEFM is generally considered to be non-thermal, the higher duty cycle and amplitude parameters described herein, particularly when applied for longer durations (e.g., longer than 5 minutes, longer than 10 minutes, etc.) may result in some heating of the tissue receiving the treatment. To avoid or ameliorate this, the apparatus may monitor the temperature of the applicator(s), e.g., one or more loops, and/or the temperature of the target tissue or adjacent tissue (including surface, e.g., skin, tissues).

Any appropriate thermal sensor may be used, including thermistor and/or non-contact sensors, such as infrared sensors. The sensor data may be used as part of a control loop by the controller (e.g., by the thermal sub-system) to continuously monitor the tissue before and during the application of the PEMF treatment. The control loop may adjust the parameters to a lower duty cycle (still within the range of effective parameters described herein, such as greater than 4% duty cycle and between 0.2 G to 1.0 G amplitude) of a treatment dose, and/or to length the delay to a longer delay between treatment doses. In some examples shorter (1 minute, 2 minute, 3 minute, 4 minute, 5 minute, etc.), doses may be applied more frequently instead of longer, less frequent dosing. The total energy applied within a day or week may be the same, but the dosing may be distributed to prevent excessive heating of the tissue.

In any of these apparatuses the cooling sub-system may also or alternatively be configured to cool the tissue of the patient before, during and/or after treatment is applied to avoid excessive heating. Cooling may also or alternatively be applied to the applicator(s).

In one example, cooling may be applied to the tissue directly by, e.g., cooling the patient's blood before or during treatment.

Any of these apparatuses may include an automatic shutdown or shutoff if the temperature of the target tissue and/or the coil loop applicators exceed a threshold. The threshold may be predefined or user selected. For example, the threshold should be selected to reduce or prevent damage to the patient. In some examples the threshold may be within +/−0.3 degrees C. of normal body temperature (e.g., within +/−0.4 degrees C., +/−0.5 degrees C., +/−0.7 degrees C., +/−1 degree C., +/−2 degrees C., +/−3 degrees C., +/−4 degrees C., +/−5 degrees C., etc.).

The PEMF parameters for reducing an inflammatory response described herein describe a range of duty cycle and amplitude for the PEMF parameters that is critical to achieve a significant reduction in inflammation; outside of this range, although a slight reduction in inflammation may be seen, the reduction may be inconsistent, and variable. For example, outside of the ranges indicated, the reduction may be seen in just some of the cytokines/chemokines involved in inflammation. Within the identified range (e.g., greater than 4% duty cycle, and having an amplitude of between 0.2 G and 1 G, or less than 1 G) a significant reduction (e.g., 25% or more) may be reliable seen in a variety of tissue and animals, including human and non-human animals. Surprisingly outside of this range the effect on inflammation may be a significant increase (rather than no effect of a decrease) in the inflammatory response. The significant increase in an inflammatory response may be desirable in some examples and thus methods of increasing an inflammatory response (and apparatuses for increasing the inflammatory response) are also described herein.

For example, a method of increasing inflammation in a patient may include: applying pulsed electromagnetic field (PEMF) energy to the patient from an applicator (e.g., an antenna, a coil, etc.), wherein the pulsed electromagnetic energy has a duty cycle of 4% or greater and an amplitude of greater than 1 G, resulting in an increase in an inflammatory response. The increase in the inflammatory response may be an increase in one or more cytokines and/or chemokines. The increase may be an increase, e.g., of 20% or more (e.g., 25% or more, 30% or more, 35% or more, 40% or more, 45% or more, 50% or more, etc.). Examples of cytokines and/or chemokines that may be increased by the application of PEMF within the range of parameters identified herein may include one or more of: Macrophage Inflammatory Protein 1 alpha (MIP-1α), Interleukin 1 beta (IL-1β), Interferon gamma-induced protein 10 (IP-10), Macrophage inflammatory protein-1B (MIP-1β), Interleukin 6 (IL-6), tumor necrosis factor alpha (TNF-α), and Monocyte Chemoattractant Protein-1 (MCP-1).

For example, a method of increasing an inflammatory response (e.g., inflammation) may include: applying pulsed electromagnetic energy to the patient from a coil, wherein the pulsed electromagnetic energy has a duty cycle of 4% or greater and an amplitude of greater than 1 G, resulting in an increase in the patient of 25% or more of one or more of: Macrophage Inflammatory Protein 1 alpha (MIP-1α), Interleukin 1 beta (IL-1β), Interferon gamma-induced protein 10 (IP-10), Macrophage inflammatory protein-1B (MIP-1β), Interleukin 6 (IL-6), tumor necrosis factor alpha (TNF-α), and Monocyte Chemoattractant Protein-1 (MCP-1).

Applying the pulsed electromagnetic energy may include applying the pulsed electromagnetic energy at an amplitude of between about 1 G and about 4 G (e.g., between 1 G and 5 G, between 1 G and 4.5 G, between 1 G and 3.5 G, between 1 G and 3 G, between 1 G and 2.5 G, between 1.1 G and 4 G, between 1.2 G and 4 G, more than 1.1 G, more than 1.2 G, between 1.2 G and 4 G between 1.1 G and 5 G, between 1.1 G and 3 G, between 1.1% and 2 G, etc.). Applying the pulsed electromagnetic energy may comprise applying the pulsed electromagnetic energy at an amplitude of 1.1 G or greater.

The methods of increasing an inflammatory response described herein may generally be used as a method of treating an infection, such as (but not limited to) one or more of: a bacterial, a viral and/or a parasitic infection. Any of these methods of increasing an inflammatory response may be a method of enhancing an immune response.

In some examples the methods of increasing an inflammatory response may be a method of treating a tumor. For example, any of these methods may be methods of administering a cancer vaccine or antigen to patient in need thereof in combination with application of pulsed electromagnetic energy.

Any of the methods described herein may include applying pulsed electromagnetic energy for a minimum treatment dose time/duration, such as, e.g., applying for at least 5 minutes (e.g., at least about 6 minutes, at least about 7 minutes, at least about 8 minutes, at least about 9 minutes, at least about 10 minutes, at least about 11 minutes, at least about 12 minutes, at least about 13 minutes, at least about 14 minutes, at least about 15 minutes, at least about 16 minutes, at least about 17 minutes, at least about 18 minutes, at least about 19 minutes, at least about 20 minutes, at least about 25 minutes, at least about 30 minutes, etc.). In some examples the minimum dose duration may be less than 5 minutes.

In any of the methods of increasing inflammation described herein the applied pulsed electromagnetic energy may have a duty cycle of between 4% and 20%. In any of these methods of increasing inflammation (e.g., increasing the inflammatory response), applying the pulsed electromagnetic energy may comprise increasing levels of one or more of: Macrophage Inflammatory Protein 1 alpha (MIP-1α), Interleukin 1 beta (IL-1β), Interferon gamma-induced protein 10 (IP-10), Macrophage inflammatory protein-1B (MIP-1β), Interleukin 6 (IL-6), tumor necrosis factor alpha (TNF-α), and Monocyte Chemoattractant Protein-1 (MCP-1), by 30% or more in the patient. For example, any of these methods may include detecting measuring a level of or more of: Macrophage Inflammatory Protein 1 alpha (MIP-1α), Interleukin 1 beta (IL-1β), Interferon gamma-induced protein 10 (IP-10), Macrophage inflammatory protein-1B (MIP-1(3), Interleukin 6 (IL-6), tumor necrosis factor alpha (TNF-α), and Monocyte Chemoattractant Protein-1 (MCP-1).

Applying pulsed electromagnetic energy may include applying busts of pulsed electromagnetic energy having a burst width of between about 10 msec and 500 msec (e.g., 10 msec and 450 msec, 10 msec and 400 msec, 10 msec and 350 msec, 10 msec and 300 msec, 10 msec and 200 msec, 10 msec and 150 msec, 10 msec and 125 msec, 10 msec and 100 msec, 25 msec and 500 msec, 25 msec and 400 msec, 25 msec and 300 msec, 25 msec and 200 msec, etc.).

In any of these methods and apparatuses, the pulsed electromagnetic energy may have a carrier frequency of about 27.12 MHz.

As mentioned above, the pulsed electromagnetic energy may be applied to a target tissue without increasing a temperature of the target tissue.

Applying pulsed electromagnetic energy from the coil may comprise applying the pulsed electromagnetic energy to the patient's head. Applying pulsed electromagnetic energy from the coil may comprise applying pulsed electromagnetic energy from a plurality of coils coupled to a headgear configured to be worn by the patient. In some examples, applying pulsed electromagnetic energy from the coil comprises applying pulsed electromagnetic energy from a plurality of coils that are fired independently, simultaneously, or in sequence.

Also described herein are apparatuses configured to apply PEMF to increase an inflammatory response (e.g., inflammation). For example, an electromagnetic treatment apparatus for increasing inflammation in a target tissue may include: one or more coils; a frame configured to secure the one or more applicator loops adjacent to a target tissue; and a waveform generator coupled to the one or more applicator loops, the waveform generator comprising a pulse generator configured to deliver a pulsed electromagnetic energy from the one or more coils, the pulsed electromagnetic energy having a duty cycle limited to between 4% and 20% and an amplitude limited to be between, e.g., 1 G and 4 G. The one or more applicator loops may comprise an array of applicator loops. The waveform generator may be configured to deliver the pulsed electromagnetic energy for more than 10 minutes. The frame may comprise a helmet, a brace, etc.

Also described herein are methods for treating a human or animal with an apparatus for inductive energy delivery (e.g., PEMF), the method comprising: passing pulsed bursts of high-frequency current through one or more inductive coils. The bursts of high-frequency current may be at least 1 KHz and less than 5 GHz. The burst widths of high-frequency current may be at least 0.1 microseconds and no greater than 1 second in duration. The burst pulses or repetition rates may be at least 0.1 Hz microseconds and no greater than 1000 Hz. The magnetic field strength emitted from the coil or coils may range from about 0.01 Gauss to 1 Gauss and electric field strength range from about 0.1 V/m to 40 V/m. The stimulation may reduce inflammation and reactive oxygen species production. The stimulation may be employed to promote neuroprotection of neurons, induce vasodilation, modulate the cellular activity of neurons, microglia, astrocytes, pericytes, oligodendrocytes, endothelial cells, or peripheral immune cells, or enhance mitochondrial function. The coil or coils may be placed near the head for the purpose of brain treatment. For example, the coil or coils are positioned on the head via headgear and are positioned manually or automatically. The coils may be fired independently, simultaneously, or in sequence.

Electroencephalography data may be acquired before, during, and after stimulation for the purpose of diagnosis, optimization of stimulation, coil positioning and assessment of the effects of stimulation. The coil or coils may be placed near the spinal cord, cranial nerves, or other peripheral nerves for treatment. The coil or coils may be positioned on the body via wrap or brace and allow for the adjustment of coil angle of incidence. For example, a measurement probe may be placed near the site of stimulation to collect physiological feedback data. The coil or coils may be within a biocompatible material and may be implanted under the scalp, above or within the meninges, on top or inside of the brain.

An application-enabled user interface on a phone or tablet may be used for controlling the device, performing cognitive or psychological tasks, responding to self-report questionnaires, or communication with other individuals. For example, a user interface may be integrated into a virtual reality system.

Also described herein are methods for developing an optimal stimulation paradigm for an individual comprising: artificial intelligence algorithm processing of input subject data before, during, after stimulation to generate and improve the administered stimulation parameters. The input subject data may include physician diagnosis, electroencephalography recordings, brain imaging, self-report measures, or performance on cognitive or neuropsychological tasks. The algorithm may be informed by a database of data points from healthy and ill populations including physician diagnosis, electroencephalography recordings, brain imaging, self-report measures, or performance on cognitive or neuropsychological tasks. The artificial intelligence algorithm may employ machine learning, deep learning, or neuroevolution techniques.

Stimulation may be used to treat depression, bipolar disorder, generalized anxiety disorder, social anxiety, autism, schizophrenia, post-traumatic stress disorder, attention deficit disorder and attention deficit hyperactivity disorder, substance use disorder, addiction, obsessive compulsive disorder, postpartum depression, eating disorders, sleep disorders, public speaking anxiety, stroke recovery, traumatic brain injury recovery, post-surgical recovery, post-concussion syndrome, epilepsy, spinal cord injury, Parkinson's disease, Alzheimer's disease, vascular dementia, dementia with Lewy bodies, frontotemporal dementia, Huntington's disease, fibromyalgia, chronic pain, cognitive dysfunction, Lyme disease, relapsing and progressive Multiple Sclerosis, amyotrophic lateral sclerosis, chronic fatigue.

Stimulation may be used for the enhancement of learning and memory, recall, memory capacity, attention, processing speed, coordination, relaxation, enhanced energy, sleep optimization, or transmission of information from a remote source to the nervous system.

Also described herein are systems for inductive delivery of therapy comprising: one or more coils; a coil applicator; a control module; a power supply; and a graphical user interface. The coils may be circular, figure-eight, v-shaped, or conical shaped and range from 1 micron to 30 centimeters in diameter. The coil applicator may be, for example, a headgear that is either adjustable or custom fit to the shape of the head. The coils may be manually positioned on the headgear, or an onboard motor automatically moves the coils into place before and during stimulation. In some examples, virtual reality goggles can integrate onto the headgear. The coil applicator may target the spinal cord, cranial nerves or other peripheral nerves and is secured in place by a wrap or brace.

The coil angle of incidence may be adjustable, and arrays of coils are connected in such a way as to allow them to fire sequentially. The recording electrodes may be electroencephalography electrodes built into the headgear. The recording electrodes may be cutaneous or percutaneous electrodes. The control module may be composed of memory, a microprocessor, and a software operating system that is controlled by the graphical user interface. The control module and power supply may be a self-contained unit that plugs into mains power. The control module and power supply may be a self-contained unit that is rechargeable and plugs into mains power for charging. The control module and power supply may be contained within the headgear or other coil applicators in which a rechargeable battery is part of the power supply. The control module and power supply may be contained within the headgear or other coil applicators and mains power is the power source.

The graphical user interface is an application enabled software that operates via smartphone, tablet, or virtual reality goggles. The graphical user interface allows the user to operate the device, and includes user profiles, available stimulation paradigms, chat and videoconferencing with practitioners, cognitive and neuropsychological tests, and self-report questionnaires. The coils and electronics may be miniaturized and embedded in a biocompatible material for intracranial implantation. The coil array may be powered via implantable power supply. The coil array may be powered via non-invasive induction.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 shows exemplar headgear configuration with multiple coils, electroencephalography electrodes, control module, and power supply.

FIG. 2A shows exemplar configuration, positioning, and fastener of a single coil used for treating a large region of the head. FIG. 2B shows exemplar configuration, positioning, and fastener of multiple coils used for treating large regions of the head.

FIG. 3 shows exemplar headgear configuration with multiple coils, electroencephalography electrodes, virtual reality goggle-based interface, control module, and power supply.

FIG. 4A shows an exemplar array of small diameter coils encased in a biocompatible material. FIG. 4B shows an exemplar array of coils in a biocompatible material surgically implanted on top of cortical tissue.

FIG. 5 shows components of a pulse burst neuromodulation system.

FIGS. 6A-6B illustrate an example of an experimental setup for applying pulsed electromagnetic energy (PEMF) to cells or tissue in an in vitro setting.

FIG. 7 is a graph showing that pulse electromagnetic in a variety of treatment regimens and parameters, including the specific range identified as effective for reducing inflammation (e.g., PEMF having a duty cycle of 4% or greater and an amplitude of between 0.2 G and 1 G) and the specific range identified herein as effective for increasing inflammation (e.g., PEMF having a duty cycle of 4% or greater and an amplitude of greater than 1 G) is not toxic to cells.

FIG. 8 is a graph summarizing the response to tissue (e.g., cells) following treatment with LPS for parameters including 40 msec burst, at 1 Hz duty cycle (e.g., a 4% duty cycle) showing that those PEMF parameters that also included an amplitude of between 0.2 G and about 1 G had a significantly lower inflammatory response as compared to sham control. Further, in cases in which the amplitude of the PEMF was greater than 1 G showed a significant increase in inflammation (e.g., in the inflammatory response).

FIG. 9 is a graph showing the importance of duty cycle on cytokine/chemokine activity (using MIP1a as an example chemokine).

FIG. 10 is a graph showing the importance of amplitude on cytokine/chemokine activity (using MIP1a as an example chemokine).

FIG. 11 is a graph showing a specific range of inhibition of inflammation that is characteristic of the cytokines/chemokines to PEMF as described herein, as well as a range in which inflammation was increased based on the cytokine/chemokine response. In FIG. 12, the effect of amplitude on IL1b is shown (similar to that shown in FIG. 11 for MIP1a).

### DETAILED DESCRIPTION

In general, described herein are methods and apparatuses for specifically and significantly inhibiting or enhancing inflammation in a patient, including in a specific tissue of a patient, by delivering pulsed electromagnetic field stimulation (PEMF) within a defined range of duty cycle and amplitude (described as magnetic induction, in Gauss or equivalent units). For example the methods and apparatuses described herein may be configured to reduce an inflammatory response (e.g., reduce inflammation) by applying PEMF having a duty cycle of 4% or more (e.g., greater than 4%, between 4% and 30%, between 4% and 25%, between 4% and 20%, between 4% and 18%, between 4% and 15%, between 4% and 12%, between 4% and 10%, etc.), and an amplitude (e.g., magnetic induction strength) of between about 0.2 G and about 1 G. Surprisingly, this narrow and specific range of both duty cycle and amplitude resulted in a significant reduction in inflammation, as measured by a significant reduction in cytokine and chemokine levels, in the target tissue that was treated by the PEMF. This is particularly surprising, because outside of this range of duty cycle and amplitude, little or no effect was seen, or more surprisingly, the opposite effect, e.g., an increase in inflammation as measured by cytokine/chemokine level, was seen. In particular these method of applying PEMF within the specified range (e.g., having a duty cycle of 4% or more and an amplitude of between 0.2 G and 1 G, or between 0.2 G and 0.95 G, or between 0.2 G and 0.9 G, etc.) typically reduces cytokine and chemokine (e.g., Macrophage Inflammatory Protein 1 alpha (MIP-1α), Interleukin 1 beta (IL-1β), Interferon gamma-induced protein 10 (IP-10), Macrophage inflammatory protein-1B (MIP-1β), Interleukin 6 (IL-6), tumor necrosis factor alpha (TNF-α), and Monocyte Chemoattractant Protein-1 (MCP-1)) levels by 15% or more (e.g., by 20% or more, by 25% or more, by 30% or more, by 35% or more, by 40% or more, etc.).

Further also described herein are methods of increasing an inflammatory response (e.g., inflammation) by applying PEMF within a specific range (e.g., having a duty cycle of 4% or more and an amplitude of greater than 1 G, e.g., greater than 1.1 G, greater than 1.2 G, etc.) that results in an increase in inflammation in the patient or a target tissue of the patient. For example, applying PEMF within the specific range of greater than about 1 G (e.g., greater than about 1.1 G, greater than about 1.2 G, etc.) may result in a significant increase in inflammation, which may be seen by a significant increase in cytokines/chemokines, for example, a significant (e.g., 15% or greater, 17% or greater, 20% or greater, 22% or greater, 25% or greater, 27% or greater, 30% or greater, 35% or greater, 40% or greater, etc.) in one or more of Macrophage Inflammatory Protein 1 alpha (MIP-1α), Interleukin 1 beta (IL-1β), Interferon gamma-induced protein 10 (IP-10), Macrophage inflammatory protein-1B (MIP-1β), Interleukin 6 (IL-6), tumor necrosis factor alpha (TNF-α), and Monocyte Chemoattractant Protein-1 (MCP-1).

The ranges described herein may be particularly significant because they provide a robust decrease or increase in inflammation over a broad range of different cytokines/chemokines. Although previous work had been suggestive that cytokines and chemokines levels could be modulated by the application of PEMF, replicable and robust effects had proven difficult to identify. At the ranges of duty cycle (e.g., pulse width and frequency) and amplitudes examined, small, though suggestive, effects were seen, particularly at lower duty cycles and field strengths. Further, there was little or no suggestion that the sensitivity of inflammation, including the levels of cytokines/chemokines expression in the tissue, could result in an increase in inflammation, particular at higher field strengths.

In general, described herein are methods and apparatuses (e.g., devices and systems) for the optimized delivery of energy inductively, and in particular methods and apparatuses for delivery of PEMF. In general, the devices described herein include a coil or coils placed near, on, or within a subject's head or body, a control module and power supply that is configured to deliver stimulation, an application-enable graphical user interface and employ novel pulse-burst waveforms.

Waveform configurations may vary depending on the application (e.g., increasing inflammation, decreasing inflammation, etc.) as described herein and may include high frequency carrier waves from about 1 KHz to 5 GHz (e.g., 27 MHz), burst widths from about 0.1 microseconds to 1 second, and burst pulses, or repetition rates, of around 0.1 Hz to 1000 Hz. The magnetic field strengths of these wave forms range from about 0.01 Gauss to more than 1 Gauss (more than 1.5 G more than 2 G, more than 3 G, more than 4 G, more than 5 G, etc.) and electric field strengths range from about 0.1 V/m to 500 V/m.

Any of the apparatuses described herein may include a pulse generator that may be specifically configured to apply energy (PEMF) within the defined ranges for decreasing or increasing inflammation and/or may be conjured to switch between these ranges. These apparatuses may include one or more applicators for delivering the PEMF to the patient, including to a target tissue of the patient. For example, apparatuses may include one or more coils for stimulation and these coils are of varying shapes and sizes depending on application. Coils include, but are not limited to, circular, figure-eight, v-shaped, and conical shapes and may be composed of one or more turns. Non-invasive coil diameter may range from about 2 centimeters to about 30 centimeters. Implantable single coils or arrays of coils may have coil diameters from about 1 micron to about 6 centimeters. The coils may be arranged in an array. The apparatus (e.g., a processor) may control the application of PEMF in a manner so that the emitted field(s) so not interfere. For example, an array of coils may be energized to deliver PEMF sequentially over the treatment region.

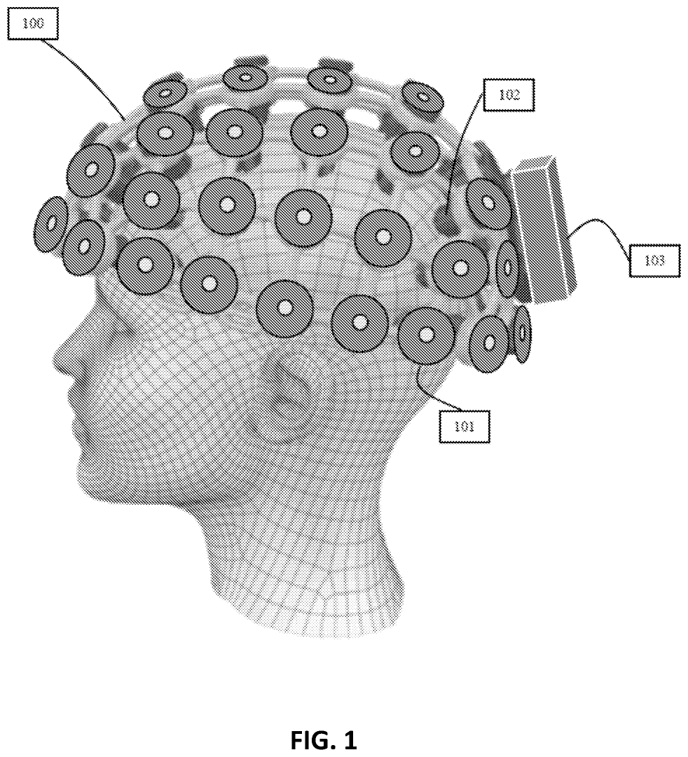

In an embodiment illustrated in FIG. 1, a head gear 100 is employed for the placement of coils 101 and administration of PEMF energy to the head (e.g., brain). Small diameter coils, for example 2-8 cm in diameter, can be used for focused application of energy in this context. Adjustable headgears can be adapted based upon a subject's head-size or, alternatively, a personalized headgear is 3D printed 100 for optimized fit. Coils are positioned on the headgear depending on application and can either stay in one position during the treatment or are moved either manually or automatically via onboard motor before and/or during treatment. Coils are fired individually, simultaneously, or in sequence. In an embodiment, the headgear contains dry or wet electroencephalography electrodes 102 in contact with the scalp. Brain activity is recorded at rest or during a cognitive or psychological task and is used in an open or closed-loop algorithm to determine and deliver optimal coil positioning and stimulation parameters. EEG signatures can be used diagnostically to determine the stimulation paradigm. For example, an EEG pattern associated with post-traumatic stress syndrome would be treated with a specific stimulation paradigm.

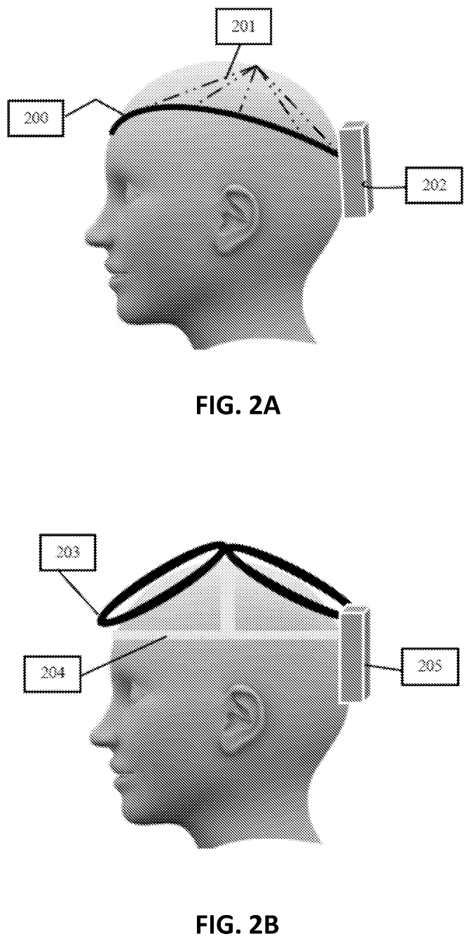

In an embodiment illustrated in FIG. 2A, larger coils, for example those greater than 10 centimeters in diameter that fit around the circumference of the head 200, can be used for applying energy to larger regions of the head (e.g., brain) and may have adjustable fasteners 201 to keep the coil in place. In an embodiment illustrated in FIG. 2B, coils large enough to treat subregions of the brain 203, can be used and have adjustable fasteners 204 to keep the coil in place. Sub-threshold field strengths would be used in these applications of the methods and apparatuses in order to reduce the risk of seizure and to achieve effects that are not dependent on depolarization. For example, sub-threshold neuromodulation would be applied to promote neuroprotection of neurons, reduce production of reactive oxygen species, reduce inflammation, induce vasodilation, modulate the cellular activity of neurons, microglia, astrocytes, oligodendrocytes, endothelial cells, and peripheral immune cells, and enhance mitochondrial function.

An aspect of the methods and apparatuses described herein is that the positioning of coils is determined based upon imaging, and/or anatomical landmarks, and/or electroencephalography (hereinafter “EEG”) recording with or without tactile or auditory cues for localization of sensory cortex. Additionally, coil positioning is determined by artificially intelligent algorithms that are described below.

Another aspect of the methods and apparatuses involves a cloud-based database of EEG recordings from healthy and ill populations that is applied in artificial intelligence, machine learning, deep learning, or neuroevolution algorithms to personalize and optimize stimulation paradigms, coil positioning, and neuromodulation outcomes.

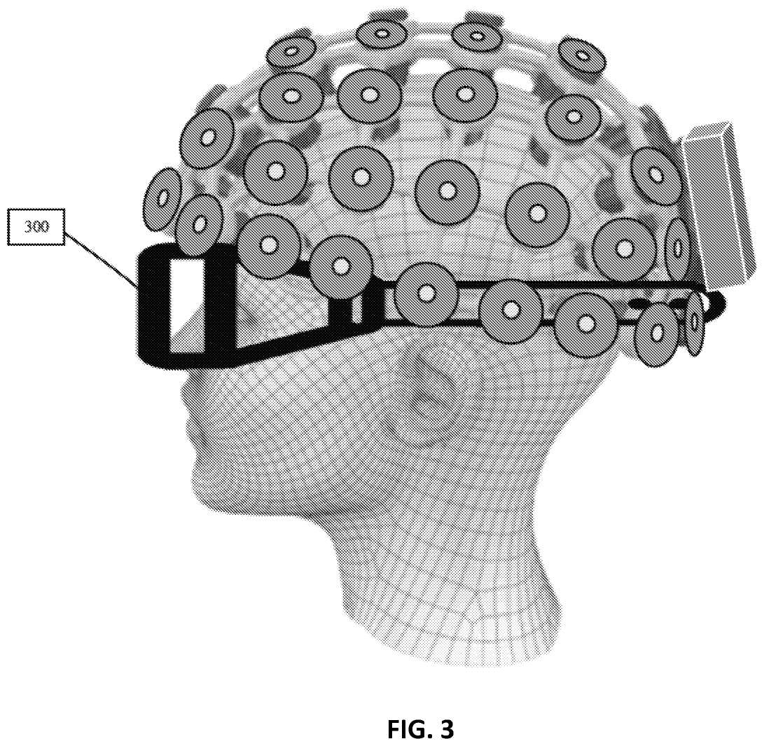

In an embodiment illustrated in FIG. 3, the headgear includes a virtual reality headset 400 built into the apparatus. The subject may operate the graphical user interface through the virtual reality portal and may also engage with a practitioner or technician via videoconference before, during, or after a stimulation session. The virtual reality interface may also be used for applying cognitive and neuropsychological tests, completing self-report measures, or assessing the effects of stimulation in real-time.

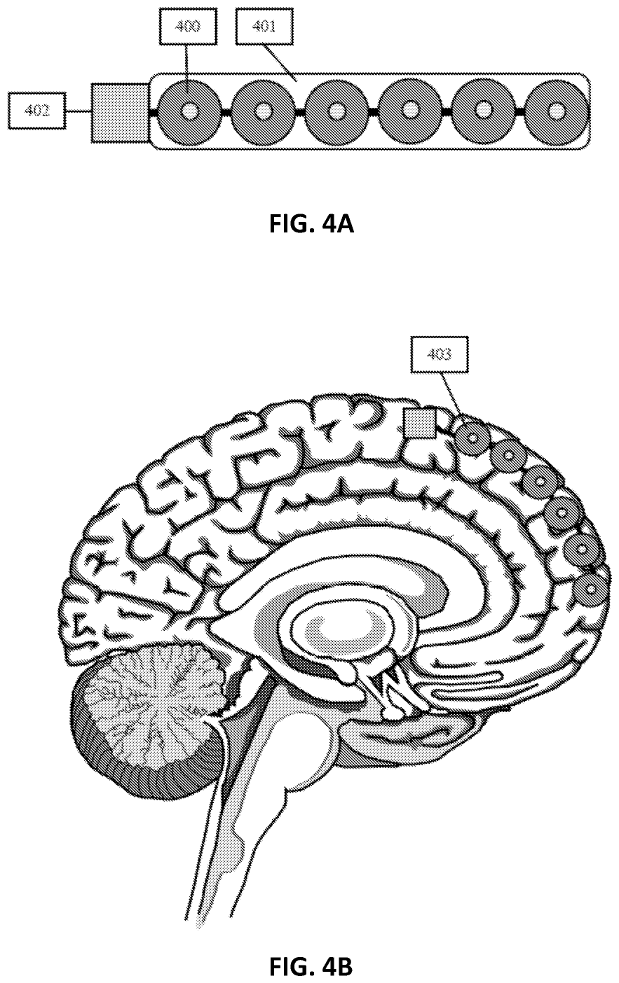

In an embodiment illustrated in FIGS. 4A and 4B, a coil or array of coils 400 is contained within biocompatible material 401 and is implanted under the scalp, above or within the meninges, on top of or inside the brain 403. The coil array provides the advantage of stimulating neuronal populations directly without the pitfalls of making electrical contact with tissue, which causes inflammation, cell death, electrode encapsulation, and other problems. Applying the energy-efficient pulse-burst method to this embodiment allows for wireless radiofrequency powering of the coils from an external control module and power supply.

An aspect of the methods and apparatuses is the control module and power supply that connect to the headgear or brace, stimulation coils, EEG, and virtual reality components. Depending on configuration, the control module and power supply may be (1) a separate unit that plugs into mains power; (2) a separate unit that is rechargeable and plugs into mains power for charging; (3) a component contained within the headgear or braces described in which a rechargeable battery serves as the source of power; (4) a component contained within the headgear or braces described in which mains power is the source of power. The control module contains onboard memory, a microprocessor and an operating system that are driven by the graphical user interface.

An aspect of the methods and apparatuses is an application-enable graphical user interface that operates via smartphone, tablet, or virtual reality headset. The graphical user interface is wirelessly connected with the control module and power supply via Bluetooth. The graphical user interface is also connected to a network that includes device user profiles and prescriptions, a database of healthy and ill EEG recordings, and a team of practitioners and technicians that are available for supervision of stimulation sessions and/or other support. The graphical user interface allows the user to activate physician-prescribed diagnostic tests or therapeutic stimulation sessions, or in non-clinical scenarios, performance-enhancement stimulation sessions. The interface also serves as a communication portal between a device user and their practitioner or performance-enhancement coach, including encrypted messaging, chat, and videoconferencing.

In addition to the treatment of inflammation (e.g., reducing or in some cases increasing inflammation), application of the pulse-burst methodology for inductive neuromodulation may have applications in psychiatry including, but not limited to, the treatment of depression, bipolar disorder, generalized anxiety disorder, social anxiety, autism, schizophrenia, post-traumatic stress disorder, attention deficit disorder and attention deficit hyperactivity disorder, substance use disorder, addiction, obsessive compulsive disorder, postpartum depression, eating disorders, sleep disorders, and public speaking anxiety.

Application of the pulse-burst methodology for inductive neuromodulation may have applications in neurology including, but not limited to, the treatment of stroke recovery, traumatic brain injury recovery, post-surgical recovery, post-concussion syndrome, epilepsy, spinal cord injury, Parkinson's disease, Alzheimer's disease, vascular dementia, dementia with Lewy bodies, frontotemporal dementia, Huntington's disease, fibromyalgia, chronic pain, cognitive dysfunction, Lyme disease, relapsing and progressive Multiple Sclerosis, amyotrophic lateral sclerosis, and chronic fatigue.

Application of the pulse-burst methodology for inductive neuromodulation may have applications in performance enhancement or wellness including, but not limited to, enhancement of learning and memory, recall, memory capacity, attention, processing speed, coordination, relaxation, enhanced energy, sleep optimization, and transmission of information from a remote source to the nervous system.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

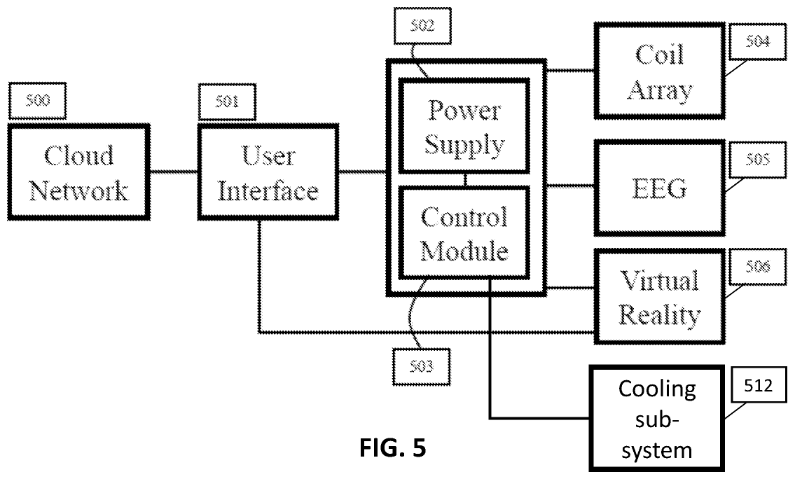

FIG. 5 schematically illustrates one example of an apparatus (e.g., system) as described herein. In this example, the apparatus includes a user interface 501 for output from one or more processors (not show) or controllers controlling the application of PEFM as described herein. In the example shown the apparatus may include a user interface that may be for communication with the user (e.g., doctor, medical technician, nurse, etc.) and/or in some cases the patient themselves. Either the user and/or the patient may include a virtual reality interface 506 for communications to/from the controller (e.g., control module 503) which may include or be coupled to a pulse generator (not shown) and power supply 502. The controller may drive the application of energy (PEMF) on the one or more applicators, such as a coil or array of coils 505. Th controller may also receive input from one or more sensors, including EEG sensors 505. As shown in FIG. 5, the entire system may be in communication with a remote (e.g., cloud 500) server or network.

The example system shown in FIG. 5 also includes a cooling sub-system 512 that may detect the temperature of the tissue being treated (or a region in thermal communication with the region being treated) and may stop the application of energy if the temperature exceeds a threshold. Alternatively or additionally, the cooling sub-system may apply cooling to the patient (e.g., to the tissue being treated and/or to a region adjacent the tissue being treated, including the skin). The cooling sub-system may also, in some examples, apply cooling to the applicator(s).

### Inflammation

The methods and apparatuses described herein may be applied to a subject (e.g., patient) to treat an indication associated with inflammation. The subject may be a human or non-human subject, such as an animal subject (e.g., vertebrate animals, including domestic animals, such as but not limited to dogs, cats, horses, pigs, sheep, cows, etc.). In general, these methods may be applied to the subject externally (e.g., non-invasively) using an applicator as described above. In some cases, the methods may include applying the treatment internally, via one or more implants or inserted applicators.

The methods and apparatuses described herein may be used prophylactically, or in in a patient diagnosed with an inflammatory disorder or disease, or both. In general, these methods may include coupling the subject to the applicator; the applicator is in turn coupled to the controller controlling the application of PEMF. The applicator may be worn by the subject externally, and may be applied against the skin of the subject directly or through a wrapping, garment, mount, frame, etc. In some examples the applicator may be applied by adhesive or hydrogel to the subject. In some examples the applicator may be part of a bed, chair, set, couch, pillow, etc. that the subject sits or lies on. More than one applicator may be used. The applicator may be coupled by one or more wires to the controller and/or pulse generator.

The controller may be controlled automatically, manually or semi-automatically. For example, the controller may receive input (and generate output) to a user such as a medical professional (e.g., nurse, doctor, etc.). The user may select the protocol to be applied from one or more (e.g., a menu) preset and/or adjustable protocols including the PEMF parameters to be applied. For example, the user may select the protocol from a menu of inflammation reducing protocols or inflammation increasing/enhancing protocols. The menu may allow the user to select based on the size and/or location on the body where the applicator is to e placed for treatment (e.g., head, arms, legs, torso, etc.).

The controller may automatically or manually (by user and/or subject input) determine the duration of the treatment to be applied. The duration of the treatment may be based on the size and/or location of the target tissue. The duration may generally be greater than a minimum treatment dose (e.g., 5 minutes or greater, 6 minutes or more, 7 minutes or more, 8 minutes or more, 9 minutes or more, 10 minutes or more, 11 minutes or more, 12 minutes or more, 13 minutes or more, 14 minutes or more, 15 minutes or more, 16 minutes or more, 17 minutes or more, 18 minutes or more, 19 minutes or more, 20 minutes or more, between 5-30 minutes, between 5-25 minutes, between 5-20 minutes, between 5-15 minutes, etc.).

The controller may monitor and adjust the parameters applied in real time during the application. For example, the controller may receive input from one or more sensors and/or inputs and may modify the applied PEMF (or the PEMF to be applied). In some examples the controller may receive input either automatically or manually from the user to adjust or to automatically adjust the PEMF applied.

For example, a method of treating a subject to reduce inflammation may include coupling a treatment application (e.g., one or more, including an array) of coils to the patient, and/or in some examples to a target tissue to be treated. In some examples the applicator may be positioned over or adjacent to the target tissue. For example, if the target tissue is a tumor or organ the one or more applicators may be positioned on the subject's body at or near the tumor or organ, including on a skin surface overlaying the tumor or organ.

The controller may then apply the PEMF using a regime in which the duty cycle is 4% or greater and the amplitude is between about 0.2 G and about 1 G (or less, such as between about 0.2 G and about 0.95 G, between about 0.2 G and about 0.9 G, etc.). The PEMF may be applied to the patient for a predetermine time period or an adjustable time period. For example, the PEMF may be applied for between 5-30 minutes. The PEMF may be maintained at the same or approximately the same level. A treatment may include applying multiple doses over a time period spanning one or more hours, days, weeks, months, etc.

In some examples the treatment may be monitored by detecting one or more indicators of inflammation. Specifically, the methods and apparatuses described herein may monitor and/or receive information on cytokines and/or chemokines. For example, one or more levels of a cytokine and/or chemokine may be examined, and this information provided, either automatically or manually, to an apparatus as described herein. In some examples, the apparatus may adjust the applied PEMF energy (e.g., adjust within the range for the duty cycle and/or amplitude). For example, if the cytokine/chemokine level(s) are higher than a threshold when attempting to reduce inflammation, the applied PEMF energy may be increased in either duty cycle and/or amplitude, within the ranges provided (e.g., for duty cycle, between about 4% and 30%, or between about 4% and 25%, etc.; for amplitude, between about 0.2 G and 1 G, between about 0.2 G and about 0.95 G, etc.). Similarly if the treatment is for increasing inflammation and the cytokine/chemokine level(s) are lower than a threshold, the applied PEMF energy may be increased in either duty cycle and/or amplitude, within the ranges provided (e.g., for duty cycle, between about 4% and 30%, or between about 4% and 25%, etc.; for amplitude the range may be 1 G or more, e.g., greater than 1 G, between about 1 G and 5 G, etc.).

In practice the methods described herein may result in changes to one or more cytokines and/or chemokines. Examples of cytokines/chemokines may include, but are not limited to pro-inflammatory cytokines/chemokines such as: G-CSF, IFN-α, IFN-β, IFN-γ, IL-11, IL-17, IL-18, IL-6, IL-8, TNF-α, TNF-β.

In general, the PEMF therapy methods and apparatuses described herein may include the application of pulsed energy within a range that is effective to recue inflammation, which may be detected by detecting a change in a marker for inflammation such as a cytokine. This reduction may be robust across more than one cytokine and may generally be applicable to multiple cytokines/chemokines.

In general, the methods and apparatuses described herein may include decreasing inflammation in the patient; this decrease may be quantified, for example, by expressing this as a decrease of a percentage (e.g., 15% or more, 20% or more, 25% or more, 30% or more, etc.) of a cytokine or chemokine in the patient. A decrease of cytokine or chemokine in the patient may include a decrease of the cytokine and/or chemokine in a target tissue of the patient. For example, cytokines, chemokines, other inflammatory proteins may be measured from the blood, plasma, serum, cerebrospinal fluid (ELISA, Flow Cytometry, Immunocytochemistry). In some examples detection of a decrease in inflammation may be detected by drawing blood and culturing whole blood or immune cells under resting conditions or after inflammatory challenge (1) to measure cytokines, chemokines, other inflammatory proteins in supernatant; or (2) to measure inflammatory gene expression using RT-PCR, DNA microarray, or RNA sequencing. In some examples, detection of a decrease in inflammation from the patient may include detecting from a biopsy of tissue or bodily fluid to measure RNA or protein for cytokines, chemokines, or other inflammatory proteins (ELISA, Flow Cytometry, Immunocytochemistry, RT-PCR). In some examples, detection from the patient (e.g., from a target tissue of the patient) may include measuring brain inflammation using PET imaging with TSPO ligand (marker of microglial inflammation) and/or measuring body temperature, redness, swelling, pain. These measurements may be quantified.

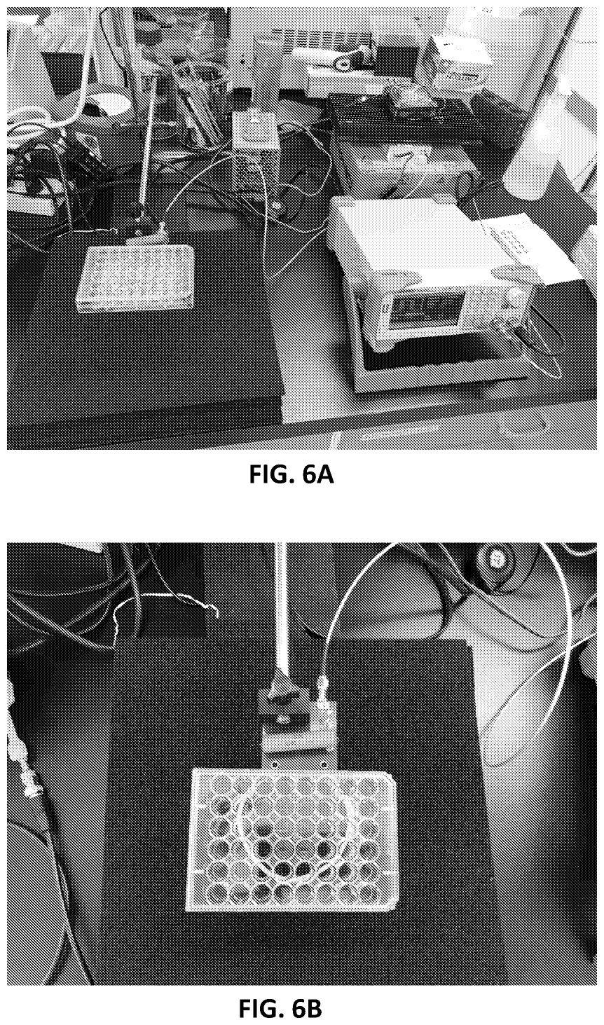

FIGS. 6A and 6B illustrate an experimental setup for examine the effects of PEMF on inflammation. FIG. 6A shows an example of an applicator coupled over a culture dish having a plurality of different wells. The wells in this example include THP-1 Cells (Human Monocyte Cell Line) that are differentiated into macrophage/microglia phenotype via incubation with phorbol myristate acetate (PMA). In some examples, approximately 200 k macrophage/microglia are included in each well, with 6 wells per plate, 16 plates (13 active waveforms, 3 sham control)

All wells pulsed with LPS (5 ng/ml) and immediately treated for 15 minutes with waveforms 1-13 or Sham on benchtop. Plates incubated with LPS for 24, before samples taken for analysis. Initially, a large range of waveforms were examined for the application of PEMF. For example, table 1 shows a set of parameters indicated by number that were examined for their effects on inflammation.

TABLE 1

| Identifier | | Amplitude | Duty | | |
|---|---|---|---|---|---|
| No. | | (G) | Cycle | Burst parameters | |
| 1 | 2.25 V/m | 0.05 G | 0.40% | 2 msec burst, 2 Hz | — |
| | | | | (Torino) | |
| 2 | 2.25 V/m | 0.05 G | 1.00% | 10 msec burst, 1 Hz | — |
| 3 | 2.25 V/m | 0.05 G | 4.00% | 40 msec burst, 1 Hz | — |
| 4 | 10 V/m | 0.24 G | 0.40% | 2 msec burst, 2 Hz | — |
| 5 | 10 V/m | 0.24 G | 1.00% | 10 msec burst, 1 Hz | — |
| 6 | 10 V/m | 0.24 G | 4.00% | 40 msec burst, 1 Hz | * |
| 7 | 10 V/m | 0.24 G | 4.00% | 1 msec burst, 40 Hz | * |
| 8 | 20 V/m | 0.47 G | 0.40% | 2 msec burst, 2 Hz | — |
| 9 | 20 V/m | 0.47 G | 1.00% | 10 msec burst, 1 Hz | — |
| 10 | 20 V/m | 0.47 G | 4.00% | 40 msec burst, 1 Hz | * |
| 11 | 50 V/m | 1.2 G | 0.40% | 2 msec burst, 2 Hz | — |
| 12 | 50 V/m | 1.2 G | 1.00% | 10 msec burst, 1 Hz | — |
| 13 | 50 V/m | 1.2 G | 4.00% | 40 msec burst, 1 Hz | ** |

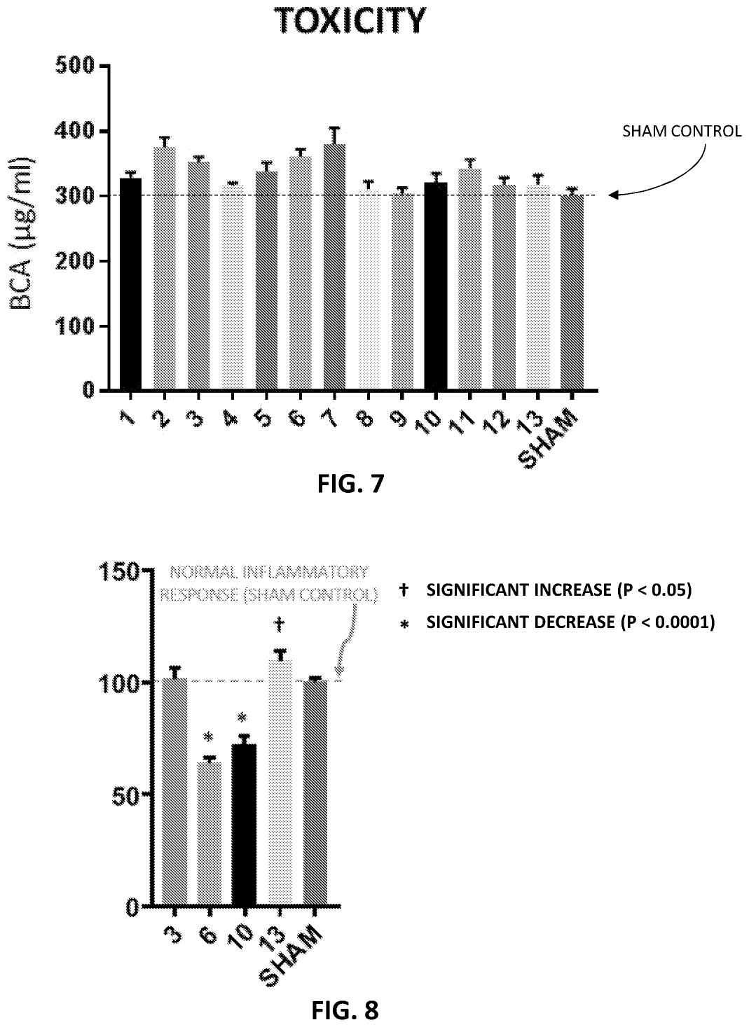

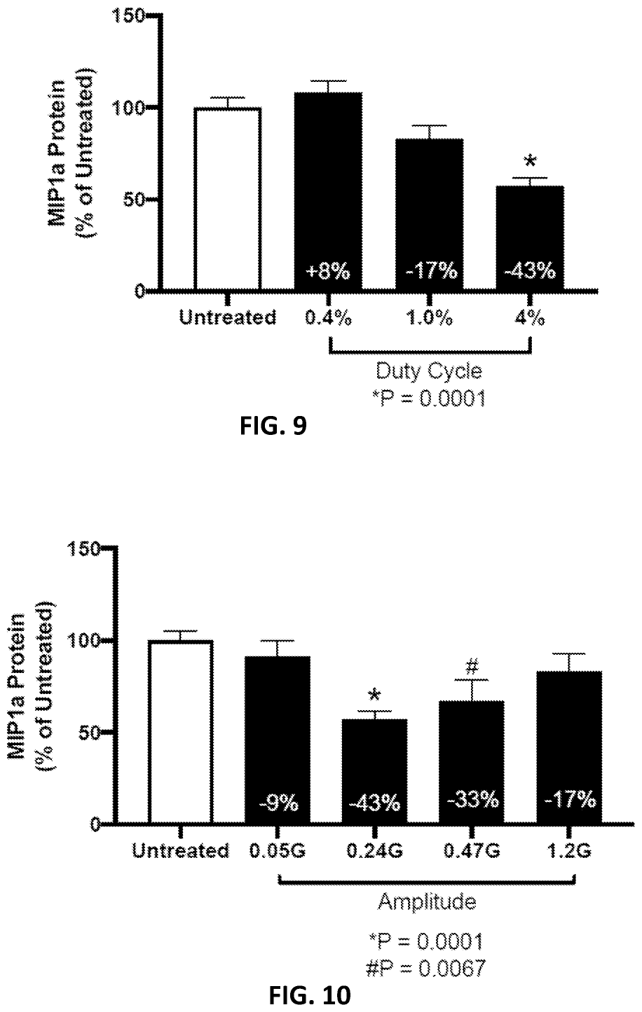

Table one shows a range of parameters spanning those that did (*) and did not significantly modulate inflammation across different cytokines/chemokines, including both inhibiting inflammation (*) and increasing inflammation (**). Other parameters within the effective ranges for degreasing and increasing inflammation were tested as well. As shown in FIG. 7, in general, the PEMF therapies described herein, including those in table 1, were not toxic to cells or tissue and did not inhibit their response to PEMF. Instead, as shown in FIG. 7, the PEMF energy applied enhanced the viability of the cells under general inflammatory conditions, such as in the presence of LPS to stimulate an inflammatory condition.

The parameters shown in Table 1, as well as others, were examined using assays on cell cultures cells (results summarized in FIG. 8 for those parameters having a duty cycle of 4% or greater). Similar results were found in preliminary animal model data.

In FIGS. 7 and 8, equal numbers of cells were loaded for each condition. After treatment and LPS exposure, BCA protein, which is proportional to and a surrogate for cell number, was measured. Compared to untreated sham controls, there was no evidence of toxicity under any EMF treatment condition and, in fact, five of the EMF conditions significantly increased cell viability after LPS exposure. In FIG. 8, “*” refers to a P≤0.05.

Thus, FIG. 8 shows an example of experiments showing that there is a reduced inflammatory response after LPS treatment is provided across a broad range of inflammatory disorders. In FIG. 8 the results of a variety of cytokines and chemokines were examined, and summarized in the graph. For example, the cytokines/chemokines were examined, including: G-CSF, IFN-α, IFN-β, IFN-γ, IL-11, IL-17, IL-18, IL-6, IL-8, TNF-α, TNF-β, MIP-1α, IL-1β, IP-10, MIP-1β and MCP-1. Note that the lowest responses tended to be for mediators where the Luminex assay has low sensitivity. More sensitive ELISA assays show more pronounced and somewhat different responses.

As described above, there is a range of duty cycle and amplitudes that are effective in reducing inflammation, as may be seen by the reduction in cytokine/chemokine level(s) across a variety of known cytokine/chemokines. Outside of this range little significant impact on inflammation is seen. For example, FIG. 10 demonstrates the importance of duty cycle on the inhibition of inflammation. FIG. 10 illustrates the dependence of the reduction in inflammation in the context of the chemokine MIP1a. In FIG. 10, the change in MIP1a protein (as a percent of untreated cells) did not become significantly different until the duty cycle was increased to about 4%.

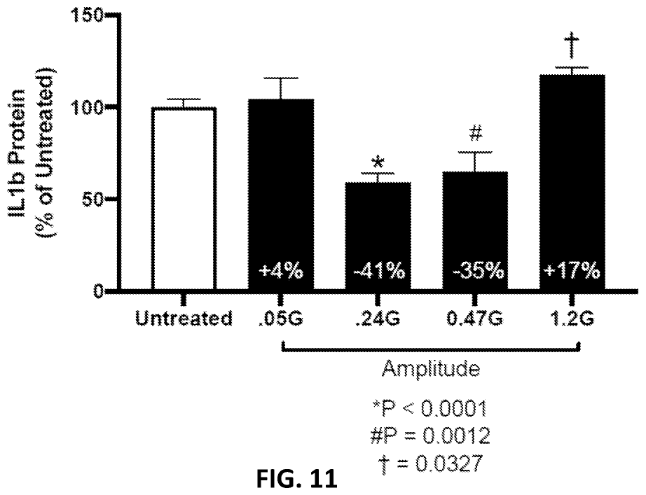

FIG. 11 demonstrates the importance of amplitude of the PEMF energy, shown in this example in the representative context of MIP1a. In FIG. 11, the specific range of amplitudes (between 0.05 G and 1.2 G) shows that at amplitudes lower than about 0.2 G no significant effect was seen on inflammation as reflected by the level of MIP1a protein as compared to untreated cells. However, and surprisingly, above about 1 G the response indicated an increase in inflammation, rather than a decrease as shown at lower amplitude levels. This same conclusion was true across a variety of cytokine/chemokine agents. In FIG. 12 the cytokine/chemokine Isi IL1b protein, which showed the same amplitude dependency. IL1b is significantly higher than untreated at 1.2 G, while 0.24 G and 0.47 G are significantly lower.

Preliminary data indicates that similar or identical trends are true in human tissue (and therefore human patients). In general, the methods of treating or preventing inflammation may result in a 15% or more (20% or more, 25% or more, 30% or more, 40% or more, etc.) reduction in cytokine/chemokine concentration, which is also surprising as a single therapeutic exposure of 15 minutes.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-32. (canceled)

33. A method of reducing inflammation in a patient, the method comprising:
applying pulsed electromagnetic energy to the patient from a coil, wherein the pulsed electromagnetic energy has a duty cycle of 4% or greater and an amplitude of between 0.2 G and 1 G, resulting in a decrease in the patient of 20% or more of a cytokine or chemokine.

34. The method of claim 33, wherein the cytokine/chemokine is one or more of:
Tumor Necrosis Factor alpha (TNF-α), Macrophage Inflammatory Protein (MIP1a), Interleukin 1 beta (IL-1β), Interferon gamma-induced protein 10 (IP-10), and Monocyte Chemoattractant Protein-1 (MCP-1).

35. The method of claim 33, wherein the cytokine/chemokine is three or more of:
Tumor Necrosis Factor alpha (TNF-α), Macrophage Inflammatory Protein (MIP1a), Interleukin 1 beta (IL-1β), Interferon gamma-induced protein 10 (IP-10), and Monocyte Chemoattractant Protein-1 (MCP-1).

36. The method of claim 33, wherein applying pulsed electromagnetic energy comprises applying for at least 10 minutes.

37. The method of claim 33, wherein the applied pulsed electromagnetic energy has a duty cycle of between 4% and 20%.

38. The method of claim 33, wherein applying the pulsed electromagnetic energy comprises decreasing levels of one or more of: Tumor Necrosis Factor alpha (TNF-α), Macrophage Inflammatory Protein (MIP1a), Interleukin 1 beta (IL-1β), Interferon gamma-induced protein 10 (IP-10), and Monocyte Chemoattractant Protein-1 (MCP-1), by 40% or more in the target tissue.

39. (canceled)

40. The method of claim 33, wherein applying pulsed electromagnetic energy comprises applying busts of pulsed electromagnetic energy having a burst width of between about 10 msec and 150 msec.

41-42. (canceled)

43. The method of claim 33, wherein applying pulsed electromagnetic energy from the coil adjacent to the target tissue comprises applying from a plurality of coils adjacent a patient's head.

44. The method of claim 33, wherein applying pulsed electromagnetic energy from the coil comprises applying pulsed electromagnetic energy from a plurality of coils coupled to a headgear configured to be worn by a patient.

45. The method of claim 33, wherein applying pulsed electromagnetic energy from the coil comprises applying pulsed electromagnetic energy from a plurality of coils that are fired independently, simultaneously, or in sequence.

46. The method of claim 33, wherein applying pulsed electromagnetic energy from the coil comprises modifying a location or energy applied by the coil based on electroencephalography data acquired before or after application of pulsed electromagnetic energy.

47. The method of claim 33, wherein the method is a method of treating a neuroinflammatory disorder including one or more of: Encephalitis, Myelitis, Meningitis, Arachnoiditis, Neuritis, Dacryoadenitis, Scleritis, Episcleritis, Keratitis, Retinitis, Chorioretinitis, Blepharitis, Conjunctivitis, Uveitis, Otitis externa, Otitis media, Labyrinthitis, and Mastoiditis.

48. The method of claim 33, wherein the method is a method of treating a Severe Acute Respiratory Syndrome coronavirus 2 (SARS-CoV-2) infection.

49. The method of claim 33, wherein the method is a method of treating a cardiovascular inflammatory disorder including one or more of: Carditis, Endocarditis, Myocarditis, Pericarditis, Vasculitis, Arteritis, Phlebitis, and Capillaritis.

50. The method of claim 33, wherein the method is a method of treating a respiratory inflammatory disorder including one or more of: Sinusitis, Rhinitis, Pharyngitis, Laryngitis, Tracheitis, Bronchitis, Bronchiolitis, Pneumonitis, Pleuritis, and Mediastinitis.

51-52. (canceled)

53. The method of claim 33, wherein the method is a method of treating one or more of: Hepatitis, Ascending cholangitis, Cholecystitis, Pancreatitis, Peritonitisg, Dermatitis, Folliculitis, Cellulitis, and Hidradenitis, Arthritis, Dermatomyositis, Myositis, Synovitis/Tenosynovitis, Bursitis, Enthesitis, Fasciitis, Capsulitis, Epicondylitis, Tendinitis, Panniculitis, Osteochondritis, (Osteitis/Osteomyelitis), Spondylitis, Periostitis, Chondritis, Nephritis, Glomerulonephritis, Pyelonephritis, Ureteritis, Cystitis, Urethritis, Oophoritis, Salpingitis, Endometritis, Parametritis, Cervicitis, Vaginitis, Vulvitis, Mastitis, Orchitis, Epididymitis, Prostatitis, Seminal vesiculitis, Balanitis, Posthitis, Balanoposthitis, Chorioamnionitis, Funisitis, Omphalitis, Insulitis. Hypophysitis, Thyroiditis, Parathyroiditis, Adrenalitis, Lymphangitis, and Lymphadenitis.

54. The method of claim 33, wherein the coil is implanted into a patient's body.

55. The method of claim 33, further comprising adjusting the pulsed electromagnetic energy based on a level of one or more of: Tumor Necrosis Factor alpha (TNF-α), Macrophage Inflammatory Protein (MIP1a), Interleukin 1 beta (IL-1β), Interferon gamma-induced protein 10 (IP-10), and Monocyte Chemoattractant Protein-1 (MCP-1).

56. (canceled)

57. A method of reducing inflammation in a target tissue, the method comprising:
applying pulsed electromagnetic energy from a coil adjacent to the target tissue for more than 10 minutes, the pulsed electromagnetic energy having a duty cycle of 4% or greater and an amplitude of between 0.2 G and 0.9 G, resulting in a decrease in the target tissue of 30% or more of one or more of: Tumor Necrosis Factor alpha (TNF-α), Macrophage Inflammatory Protein (MIP1a), Interleukin 1 beta (IL-1β), Interferon gamma-induced protein 10 (IP-10), and Monocyte Chemoattractant Protein-1 (MCP-1).

58. A method of reducing inflammation in a patient, the method comprising:
applying pulsed electromagnetic energy to the patient from a coil for 10 minutes or longer, wherein the pulsed electromagnetic energy has a duty cycle of 4% or greater and an amplitude of between 0.2 G and 1 G, resulting in a decrease in the patient of 30% or more of one or more of: Tumor Necrosis Factor alpha (TNF-α), Macrophage Inflammatory Protein (MIP1a), Interleukin 1 beta (IL-1β), Interferon gamma-induced protein 10 (IP-10), and Monocyte Chemoattractant Protein-1 (MCP-1).

59-69. (canceled)
